(19) United States
(12) Patent Application Publication
(10) Pub. No.: US 2013/0331636 A1
(43) Pub. Date: Dec. 12, 2013

(54) Magnotherapy Products

(71) Applicants: Derek PRICE, Wiltshire (GB); Nyjon ECCLES, London (GB)

(72) Inventors: Derek PRICE, Wiltshire (GB); Nyjon ECCLES, London (GB)

(21) Appl. No.: 13/965,506

(22) Filed: Aug. 13, 2013

Related U.S. Application Data
(63) Continuation-in-part of application No. PCT/GB12/50319, filed on Feb. 14, 2012.

(30) Foreign Application Priority Data
Feb. 14, 2011 (GB) 1102554.1

(51) Int. Cl. A61N 2/06

(52) U.S. Cl. CPC A61N 2/06
USPC 600/9

(57) ABSTRACT

The present invention relates to the use of magnets in the treatment of human disorders that are caused by or contributed to by Autonomic Nervous System imbalance and include menopause symptoms wherein the magnetic product comprises a magnet having positive and negative poles, the magnet having an additional metallic directional plate which distorts the magnetic field distribution around the magnet so as to attenuate the magnetic field in the vicinity of the positive pole of the magnet.

### PRIOR RELATED APPLICATIONS

This application is a Continuation-in-part of, and claims priority to, International Application PCT/GB2012/050219, filed on Feb. 14, 2012, which claims priority to GB Application 1102554.1, filed Feb. 14, 2011. Both applications are expressly incorporated by reference in their entirety.

### FEDERALLY SPONSORED RESEARCH STATEMENT

Not applicable.

### REFERENCE TO MICROFICHE APPENDIX

Not applicable.

### FIELD OF THE DISCLOSURE

The present invention relates to the use of magnotherapy products, i.e. products including one or more magnets, in the treatment of disorders related to autonomic nervous system imbalance, in particular where there is a sympathetic nervous system dominance and/or parasympathetic nervous system insufficiency. Such imbalance can lead to stress and anxiety, recovery and repair after injury or illness, heart palpitations, vasospasm (causing reduced blood flow), digestive disorders, reduced insulin output, erectile dysfunction (Impotence) and prostatic hypertrophy in men, and increased menopause symptoms in women.

### BACKGROUND OF THE DISCLOSURE

Magnotherapy describes the practice of placing magnets adjacent an animal or human body in order to alleviate symptoms or to enhance performance of the animal or human. The mechanism by which magnotherapy works has to date not been understood or confirmed by the scientific community. A discussion of magnotherapy can be found in the book “Magnetic Health, Modern Day Healing with Magnets” by D R Price (ISBN 0953679705).

The autonomic nervous system (ANS or visceral nervous system) is the part of the peripheral nervous system that acts as a control system functioning largely below the level of consciousness, and controls visceral functions. The ANS affects heart rate, digestion, respiration rate, salivation, perspiration, diameter of the pupils, micturition (urination), and sexual arousal. Whereas most of its actions are involuntary, some, such as breathing, work in tandem with the conscious mind.

Almost all bodily functions are dependent on the functioning of the autonomic nervous system—from the cardiovascular system, the gastrointestinal tract, the evacuative and sexual organs, to the regulation of temperature, metabolism and tissue defence. Balanced functioning of this system is an important basis of our life and well-being. It is classically divided into two subsystems: the parasympathetic nervous system (PNS) and sympathetic nervous system (SNS).

With regard to function, the SNS is responsible for the body's fight-or-flight/stress reaction e.g. increased heart rate to pump blood to muscles for fight or flight and pupil dilatation to allow maximum light to reach the retina thus optimising visual response in the face of a perceived threat. The PNS, is involved in rest and regulation and digestion and tends to produce the opposite effect to the SNS; it slows the heart and constricts the pupils.

ANS innervation is divided into sympathetic nervous system and parasympathetic nervous system divisions. The sympathetic division has thoracolumbar “outflow”, meaning that the neurons begin at the thoracic and lumbar (T1-L2) portions of the spinal cord. The parasympathetic division has craniosacral “outflow”, meaning that the neurons begin at the cranial nerves (CN3, CN7, CN 9, CN10) and sacral (S2-S4) spinal cord. FIG. 1. shows the contrasting actions of the SNS and PNS as referred to above and FIG. 2. shows the positional outflow of the SNS and PNS nerve roots form the spinal cord as referred to above.

The inventors have surprisingly found that it is possible to influence the balance of the SNS and PNS using magnotherapy. Thus, what is needed is a magnotherapy device and method of use to correct imbalances in the ANS.

### SUMMARY OF THE DISCLOSURE

A first aspect of the invention relates to the use of a magnotherapy product to treat disorders related to autonomic nervous system imbalance. In particular, the invention relates to the use of a magnotherapy product to reduce the effect of the sympathetic nervous system and/or to stimulate or increase the effect of the parasympathetic nervous system. Conditions caused by imbalance of the autonomic nervous system include stress and anxiety, heart palpitations, vasospasm, digestive disorders including irritable bowel syndrome, reduced insulin output, diabetes, increased menopausal symptoms, erectile dysfunction, prostatic hypertrophy, and reduced repair or recovery after injury. The treatment of one or more or all of these conditions is provided by the invention, as is the treatment of any other condition in which there is an autonomic nervous system imbalance.

The magnotherapy product preferably comprises a magnet having positive and negative poles, the magnet having an additional metallic directional plate which distorts the magnetic field distribution around the magnet so as to attenuate the magnetic field in the vicinity of the positive pole of the magnet.

The first aspect of the invention also provides a method of reducing symptoms of autonomic nervous system imbalance by applying a magnetic field to a human body wherein the magnetic product comprises a magnet having positive and negative poles, together with an additional metallic directional plate which distorts the magnetic field distribution around the magnet so as to attenuate the magnetic field in the vicinity of the positive pole of the magnet. The method may relate to increasing PNS activity or reducing SNS activity.

The magnetic field may be applied to a particular part of the human body, usually selected according to the condition to be treated. For example, the magnetic field may be applied to the pelvic or sacral region or to the cervical (neck) region.

It is preferably that the magnotherapy product is for increasing the activity of, or the effects of the activity of the PNS. Effects of stimulating the PNS include: stress and anxiety relief, enhanced speed of recovery and repair from illness and/or injury, reduced palpitations, improved blood supply due to dilatation of blood vessels, improved digestion by stimulating enzyme secretions, improved insulin output, improved erectile function by direct stimulation of erection and reduction of prostatic hypertrophy.

The magnotherapy product or magnetic field may be selected according to the condition to be treated. The ranges of magnetic strength are optimised for clinical effect while balancing the requirements of magnet size, weight and attraction of ferrous material.

Use of the magnetic product to correct ANS imbalance, particularly to stimulate the PNS, eliminates the requirement for drugs, anti-stress or anti-anxiety drugs, may therefore reduce the overall cost of treatment of a subject. Furthermore, there is no need to determine the level of drugs required for a given subject. The use of the magnetic product also reduces and/or eliminates the risk of side effects associated with such drugs.

The magnetic product is preferably for use on humans.

In some embodiments, the magnetic product is applied to the body for at least 2, 4, 6, 8, 10, 12, 14, 16, 18, 20, 22 or 24 hours per day. Furthermore, the magnetic product may be applied to the body for from 7 to 28 days per month, for example 7 to 14 days, 7 to 21 days, 14 to 21 days, 14 to 28 days or 21 to 28 days per month.

A magnetic product may comprise a single magnet. However, the magnetic product may comprise more than one magnet, for example 2, 3, 4, 5, or 6 magnets.

The strength of magnet may be varied in accordance to the condition being treated. For example, the strength may be above 750 G, for example from 750 G to 5,000 G, such as from 800 G to 3,600 G as measured at the surface of the magnetic product.

In some embodiments, the magnet has a strength of about 2,000 G, for example from about 1,800 G to about 2,800 G. The magnet may comprise a directional plate to enhance and focus the magnetic field towards the user. The ranges disclosed are optimised for clinical effect while balancing the requirements of magnet size, weight and attraction of ferrous material.

In some embodiments, one or more of the magnets comprises neodymium.

In one embodiment of the current disclosure, the magnotherapy product may be used to treat or reduce or prevent the symptoms of menopause and perimenopause. The magnotherapy product can be worn in the pelvic region or the cervical region, depending on the symptoms being targeted. The magnotherapy product can comprise one or more magnets comprising neodymium, e.g. an iron neodymium-boron magnet, with a strength of 800 G to 3,600, preferably 1,800 G to about 2,800 G, and most preferably 2700 G, measured at the magnet surface. A directional plate can also be included in the magnotherapy product to increase the magnetic power of the positive pole of the magnet. Said magnotherapy product can be worn for at least 2, 4, 6, 8, 10, 12, 14, 16, 18, 20, 22 or 24 hours per day for optimal treatment.

In yet another embodiment, a magnotherapy product comprising one or more iron neodymium-boron magnets, each with a strength of 2700 G and an optional directional plate for increasing the magnetic power of the positive pole of the magnet, wherein said magnotherapy device can be worn for at least 2, 4, 6, 8, 10, 12, 14, 16, 18, 20, 22 or 24 hours per day in the pelvic region of a patient suffering from menopause.

In another embodiment, a magnotherapy product comprising one or more iron neodymium-boron magnets, each with a strength of 2700 and an optional directional plate for increasing the magnetic power of the positive pole of the magnet, wherein said magnotherapy device can be worn for at least 2, 4, 6, 8, 10, 12, 14, 16, 18, 20, 22 or 24 hours per day in the pelvic region of a patient suffering from perimenopause.

The use of the word “a” or “an” when used in conjunction with the term “comprising” in the claims or the specification means one or more than one, unless the context dictates otherwise.

The term “about” means the stated value plus or minus the margin of error of measurement or plus or minus 10% if no method of measurement is indicated.

The use of the term “or” in the claims is used to mean “and/or” unless explicitly indicated to refer to alternatives only or if the alternatives are mutually exclusive.

The terms “comprise”, “have”, “include” and “contain” (and their variants) are open-ended linking verbs and allow the addition of other elements when used in a claim.

The phrase “consisting of” is closed, and excludes all additional elements.

The phrase “consisting essentially of” excludes additional material elements, but allows the inclusions of non-material elements that do not substantially change the nature of the invention.

The following abbreviations are used herein:

| ABBREVIATION | TERM |
|---|---|
| ANS | Autonomic nervous system |
| PNS | Parasympathetic nervous system |
| SNS | Sympathetic nervous system |

### BRIEF DESCRIPTION OF THE DRAWINGS

FIG. 1. Shows the contrasting actions of the SNS and PNS

FIG. 2. Shows the positional outflow of the SNS and PNS nerve roots form the spinal cord.

FIG. 3. Shows the PNS to SNS ratio before and after (1 month) wearing of the magnetic device.

FIG. 4. Shows SNS activity before exposure to the magnetic device.

FIG. 5. Shows SNS activity in menopausal and perimenopausal women before exposure to the magnetic device.

FIG. 6. Shows a magnotherapy product for use in accordance with one embodiment of the invention.

FIG. 7 shows the change in supine HF/LF ratio before and after using the magnotherapy device.

FIG. 8 shows the change in resting pulse rate seen by device users.

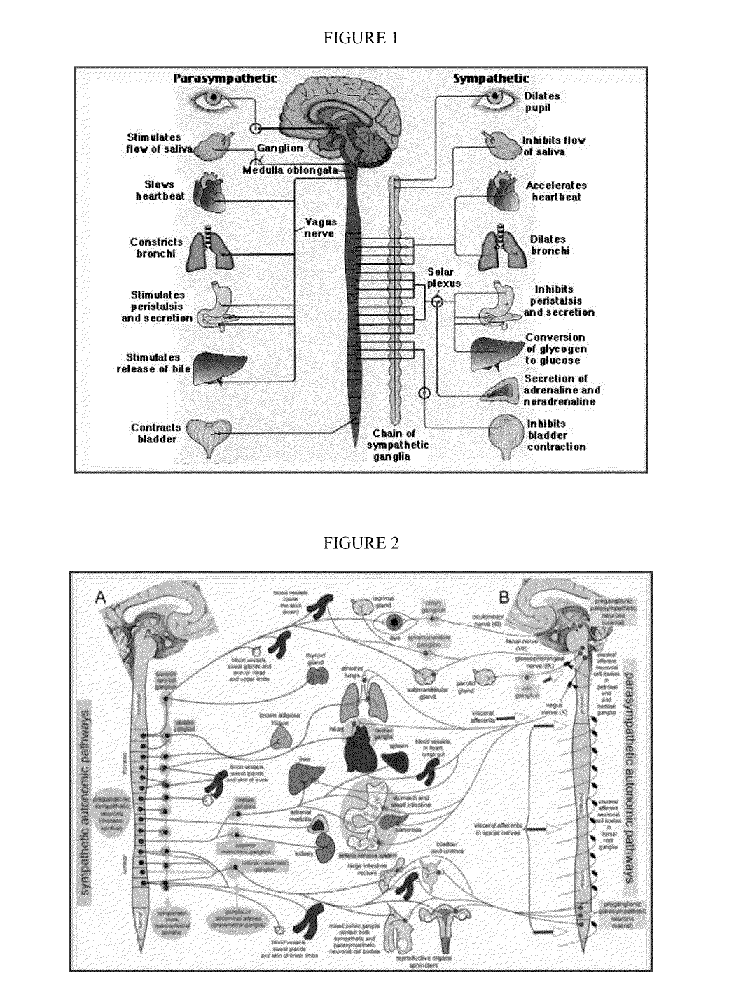

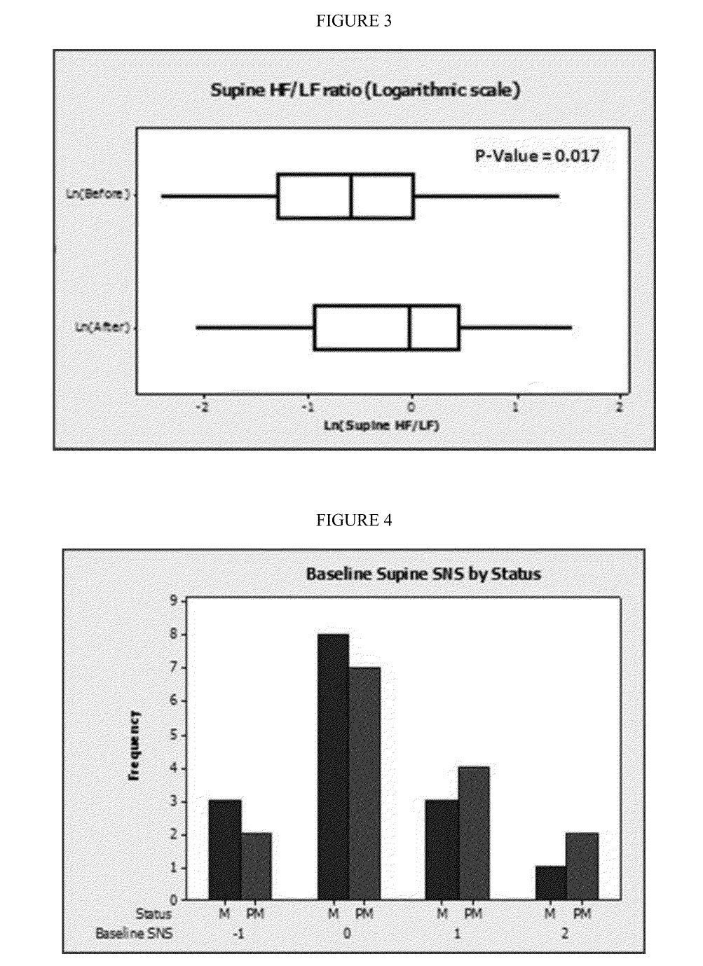

### DETAILED DESCRIPTION

The disclosure provides novel methods of treating conditions with sympathetic nervous system (SNS) dominance and/or parasympathetic nervous system (PNS) deficit using magnotherapy.

The invention will now be described in detail by way of example only. However, this is exemplary only, and the invention can be broadly applied to all disorders related autonomic nervous system imbalances. The following examples are intended to be illustrative only, and not unduly limit the scope of the appended claims.

Experimental Details

Wearing of the magnetic device has been shown to lead to changes in the ratio of PNS to SNS activity. The inventors had previously identified that the magnetic device was useful for treating menopause. The inventors hypothesised that some of the menopause symptoms that can be treated using the device are linked to ANS imbalance. The inventors conducted measurements of the ANS in 35 women, by measuring Heart Rate Variability before and after using the magnetic device. In order to investigate this hypothesis, the inventors studied the ANS activity in 35 women with menopause symptoms. The inventors identified the sweats/hot flushes associated with menopause as being the symptom most likely to be related to ANS imbalance, and so all volunteers had to have hot flushes as one of their menopause symptoms.

A recognised non-invasive method for measuring ANS activity is by Heart Rate

Variability (HRV). Close inspection and measurement of the heart rhythm of the volunteers revealed that the heart beat from one to the next is not even. The measured time between each beat varies beat to beat. This variation is a reflection of a tonic influence of the SNS (speeding the heart) and PNS (slowing the heart down). Measuring HRV is therefore a convenient way of measuring ANS activity. A 5-6 minute heart rhythm recording was taken from which computer analysis was able to derive quantitative measurements of both SNS and PNS activity.

The volunteers were treated with a magnetic device referred to as LadyCare. This device is exemplified in FIG. 6 and discussed in more detail below. FIG. 4 shows SNS activity in the volunteers before the use of LadyCare, wherein M=Menopausal women, and PM=Perimenopausal women. The zero point is neutral SNS activity so the bar chart indicates a slight tendency of SNS dominance in these women.

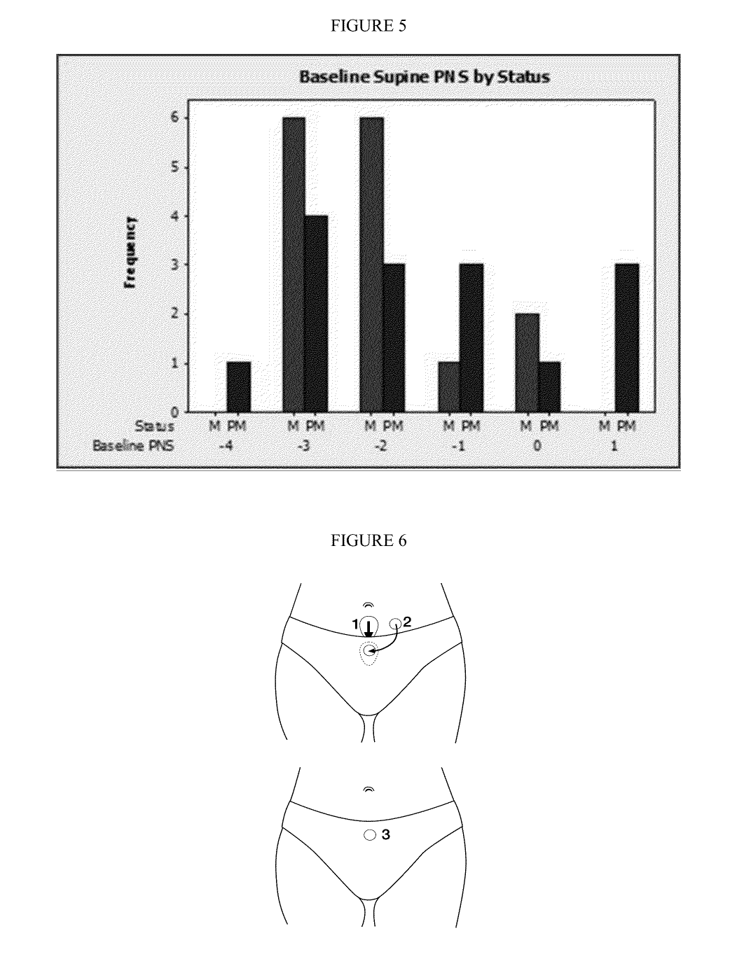

FIG. 5 shows PNS activity in the same groups of women as FIG. 4. The zero point is neutral PNS activity. In the perimenopausal group, and more so the menopausal group, there is a deficit in PNS activity. Effectively this would result in a preponderance of SNS activity over PNS activity and is a logical explanation for the occurrence of hot flushes in menopausal and perimenopausal women. This is the first report of a clear autonomic nervous system imbalance in menopause.

Autonomic Nervous System Data

35 women with menopause symptoms that had to include hot flushes were enrolled in the study. The aim was to measure ANS activity before and after exposure to the magnetic device. ANS activity was measured by Heart Rate Variabilty (HRV) using the Nerve Express software program. Menopause symptoms (23 symptom questionnaire as used previously in the inventors' previous menopause survey) were also recorded on the same 0 to 5 rating scale for each symptom, as previously. HRV and questionnaire measurements were conducted at the start and then 1 month and 3 months later. Some of the volunteers were perimenopausal and not menopausal and so these were separated for data analysis. The key findings were:

Menopausal and perimenopausal woman were found to have a slight Sympathetic (SNS) dominance.

Menopausal and perimenopausal woman were found to have a marked Parasympathetic (PNS) deficit.

The magnetic device was found to shift this ANS imbalance to a more favourable position by increasing that proportion of PNS activity

The above correction in ANS imbalance is a plausible explanation for the observed benefits of the magnetic device to relieve menopause symptoms.

The Magnotherapy Device.

The magnetic device, LadyCare, used in the presently described experiments was designed for attachment to the underwear by magnetic force. LadyCare is plastic coated and is comprised of two parts. As exemplified by FIG. 6, the pear-shaped piece (1) is worn inside of the ladies' underwear, directly against the pelvis, approximately four inches below the navel. The LadyCare contains a 20-mm×5-mm iron neodymium-boron magnet within the pear-shaped piece (1).

The second part is a circular plastic case (2) that contains a 20-mm×1-mm iron neodymium-boron magnet with a 20-mm×2-mm 400-grade stainless steel directional plate adherent to its outside. This second part is positioned on the outside of the underwear (as shown in FIG. 6) attaching securely with the force of the magnetic field (manufacturer's patent) (3). This directional plate increases the magnetic power of the north pole (standard scientific notation) of the magnetic field into the body to 2700 gauss (surface measurement made by manufacturer at the body surface of the magnet (both parts)) using a gauss meter. While the device in FIG. 6 is positioned under the navel, other positionings are possible. For instance, the device can be placed to the left or right side of pelvic region. The present disclosure is not limited to the LadyCare device or a device for treatment of peri- and menopausal patients. Other magnetic containing devices located at various outflows are possible to treat a variety of disorders that relate to the autonomic nervous system. d

Effect of Wearing the Magnotherapy Device

There is evidence of a significant increase in HF/LF ratio (a measurement of heart rate variability) i.e the ratio of parasympathetic activity to sympathetic activity after wearing the magnotherapy device. This was evident after 1 month of wearing the device and also after 3 months. Tables 1 and 2 below show that this represents a 66% change in ratio in favour of parasympathetic activity.

TABLE 1
Paired T for LnBefore-LnAfter

| | N | Mean | StDev | SE Mean |
|---|---|---|---|---|
| LnBefore | 30 | −0.685 | 0.905 | 0.165 |
| LnAfter | 30 | −0.235 | 0.944 | 0.172 |
| Difference | 30 | −0.450 | 0.969 | 0.177 |

T-Value = −2.54
P-Value = 0.017
95% CI for mean difference: (−0.812, −0.088)

TABLE 2
Analysis of variance for responder

| | | | | Adj | Adj | | |
|---|---|---|---|---|---|---|---|
| Source | DF | Seq | SS | SS | MS | F | P |
| Status | 1 | 1.9365 | 4.2253 | 4.2253 | 5.12 | 0.032 | |
| Responder | 1 | 0.5238 | 0.7035 | 0.7035 | 0.85 | 0.364 | not |
| | | | | | | | significant |
| Status*Re- | 1 | 3.3325 | 3.3325 | 3.3325 | 4.04 | 0.055 | borderline |
| sponder | | | | | | | |
| Error | 26 | 21.4426 | 21.4426 | 0.8247 | | | |
| Total | 29 | 27.2353 | | | | | |

There is some weak evidence that effect of menopausal status is dependent upon whether or not the subject is classified as a responder (p=0.055, borderline significant).

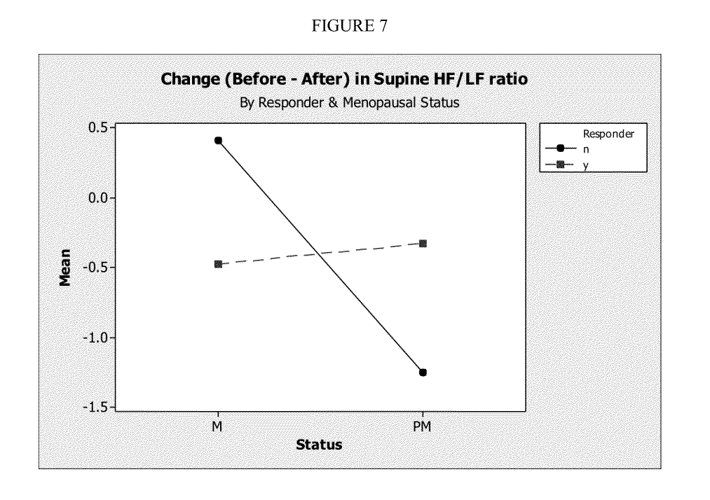

This is also shown in FIG. 7, which illustrates that those with a positive response show a different autonomic nervous system reaction than those who do not respond. This adds further weight to the conclusion that the autonomic nervous system change is the likely mechanism of action of the magnetic device as applied.

Change in Resting Pulse Rate

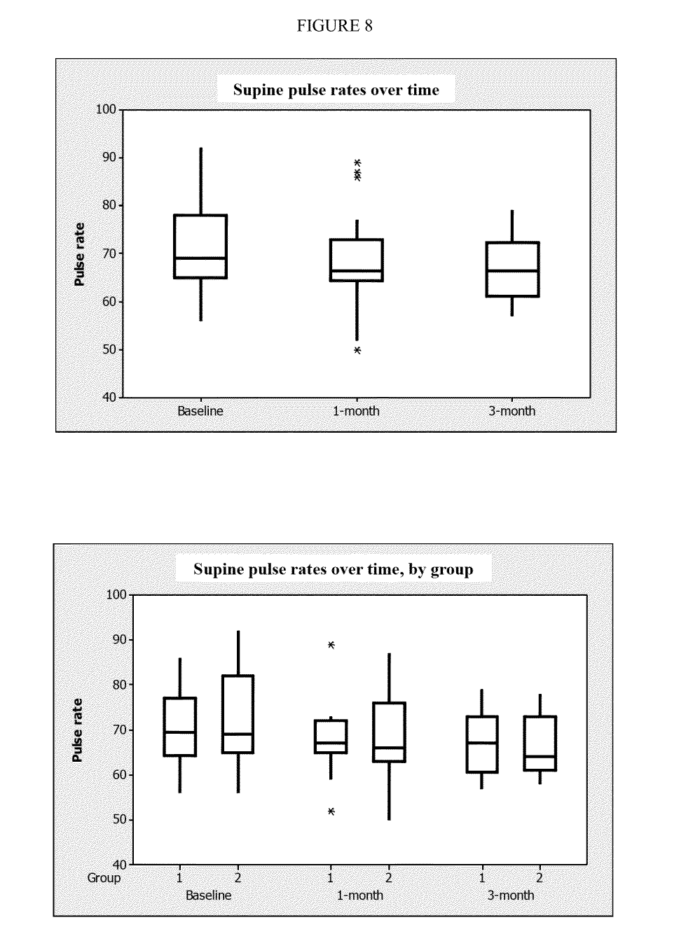

Consistent with a reduction in sympathetic nervous system activity and/or increase in parasympathetic activity was the highly statistically significant reduction in resting (supine) pulse rate seen in both menopausal and perimenopausal subjects at 1 month and 3 months after wearing the magnetic device (see FIG. 8 and Tables 3 and 4 below).

TABLE 3
Supine pulse rates, Mean (Standard Deviation)

| | Group 1, Menopausal | Group 2, Peri-menopausal |
|---|---|---|
| Time point | (n = 16) | (n = 15) |
| Baseline | 69.9 (7.8) | 71.5 (10.9) |
| 1-Month | 67.5 (8.2) | 67.7 (10.2) |
| 3-Month⁺ | 67.0 (7.4) | 66.6 (7.2) |

⁺n₁ 13, n₂ = 7

A repeated measures analysis of variance indicates that there is evidence of a statistically significant change in pulse rates over time (p=0.013), with the change being independent of group (menopausal) status:

TABLE 4
Analysis of variance for supine

| Source | DF | Seq SS | Adj SS | Adj MS | F | P |
|---|---|---|---|---|---|---|
| Time | 2 | 203.80 | 231.74 | 115.87 | 4.82 | 0.013 |
| Group | 1 | 20.02 | 6.22 | 6.22 | 0.26 | 0.613 |
| Time*Group | 2 | 12.57 | 24.92 | 12.46 | 0.52 | 0.599 |
| Subject(Group) | 29 | 4826.34 | 4826.34 | 166.43 | 6.92 | 0.000 |
| Error | 46 | 1106.71 | 1106.71 | 24.06 | | |
| Total | 80 | 6169.43 | | | | |

Pulse rates at 1 month post (p=0.016) and 3-months post (p=0.004) were significantly lower than baseline levels. Thus showing an improvement.

What is claimed is:

1. A magnetic product for use in the treatment of conditions in which Sympathetic Nervous System (SNS) dominance and/or Parasympathetic Nervous System deficit is seen, wherein the magnetic product comprises a magnet having positive and negative poles, the magnet having an additional metallic directional plate which distorts the magnetic field distribution around the magnet so as to attenuate the magnetic field in the vicinity of the positive pole of the magnet.

2. A magnetic product according to claim 1, wherein the product is used on a human or animal body for the treatment of stress and anxiety, heart palpitations, circulatory disorders where there is reduced circulation, digestive disorders including IBS, insulin deficiency (diabetes), benign prostate hypertrophy, erectile dysfunction or for enhancing speed of recovery, menopause, perimenopause, and repair after illness or injury.

3. A method of treating and/or reducing and/or preventing symptoms associated with conditions in which Sympathetic Nervous System (SNS) dominance and/or Parasympathetic Nervous System deficit is seen, comprising applying a magnetic product to a human or animal body wherein the magnetic product comprises a magnet having positive and negative poles, the magnet having an additional metallic directional plate which distorts the magnetic field distribution around the magnet so as to attenuate the magnetic field in the vicinity of the positive pole of the magnet.

4. A method according to claim 3, wherein said magnetic product is for application to a human.

5. A method according to claim 4, wherein said magnetic product is applied to or is for application to the pelvic or the cervical (neck) region of a human.

6. A method according to claim 3, wherein said magnetic product is applied to or is for application to the body for at least 2, 4, 6, 8, 10, 12, 14, 16, 18, 20, 22 or 24 hours per day.

7. A method according to claim 6, wherein said magnetic product is applied to or is for application to the body for 7 to 28 days, 7 to 14 days or 7 to 21 days.

8. A method according to claim 3, wherein said magnetic product comprises a plurality of magnets.

9. A method according to claim 8, wherein said magnetic product comprises four magnets.

10. A magnetic product according to claims 1, wherein one or more of said magnets has a strength of about 1,800 G to about 2,800 G.

11. A magnetic product according to claims 1, wherein one or more of said magnets comprises neodymium.

12. A magnetic product according to claim 2, wherein said body is a human.

13. A method of treating and/or reducing and/or preventing symptoms associated with stress and anxiety, heart palpitations, circulatory disorders where there is reduced circulation, digestive disorders including IBS, insulin deficiency (diabetes), benign prostate hypertrophy, erectile dysfunction or menopause comprising applying a magnetic product to a human body wherein the magnetic product comprises a magnet having positive and negative poles, the magnet having an additional metallic directional plate which distorts the magnetic field distribution around the magnet so as to attenuate the magnetic field in the vicinity of the positive pole of the magnet.

14. The method of claim 13, wherein menopause is treated by applying said magnetic product to the pelvic region.

15. The method of claim 14, wherein said magnetic product has a strength of about 1,800 G to about 2,800 G.

16. The method of claim 14, wherein said magnetic product has a strength of about 2,700 G.

17. The method of claim 16, wherein said magnet further comprises neodymium.

18. The method of claim 16, wherein said magnetic product is worn for at least 2, 4, 6, 8, 10, 12, 14, 16, 18, 20, 22 or 24 hours a day.
